(19) United States
(12) Patent Application Publication
(10) Pub. No.: US 2016/0106851 A1
(43) Pub. Date: Apr. 21, 2016

(54) Groundbreaking Platform Technology for Specific Binding to Necrotic Cells

(71) Applicant: HQ Medical Netherlands B.V., Rotterdam (NL)

(72) Inventors: Markwin Hendrik Maring, Rotterdam (NL); Clemens Waltherus Gerardus Lowik, Rotterdam (NL); Ermond Reijer Van Beek, Rotterdam (NL)

(21) Appl. No.: 14/820,386

(22) Filed: Aug. 6, 2015

Related U.S. Application Data
(63) Continuation of application No. PCT/NL2014/050074, filed on Feb. 6, 2014.
Application No. PCT/NL2014/050074 is a continuation of application No. PCT/NL2013/050067, filed on Feb. 6, 2013.
Application No. PCT/NL2013/050067 is a continuation of application No. PCT/NL2013/050064, filed on Feb. 6, 2013.

(30) Foreign Application Priority Data
Aug. 6, 2013 (NL) 2011274

(51) Int. Cl. A61K 47/48; A61K 49/00; G01N 33/574

(52) U.S. Cl. CPC A61K 47/4813; G01N 33/574; A61K 49/0032; G01N 2510/00

(57) ABSTRACT

A system comprising a targeting molecule for binding necrotic cells; to the system for use as a medicament; to use of the system for diagnosis; to the system for use in treatment of diseases involving necrotic cell death, to a dosage comprising the system; to a method for determining localization of the composition within a sample using said system; and, to a method for treatment of cancers and/or diseases involving necrotic cell death using said system. In particular the invention relates to compositions targeting necrotic cells.

### CROSS-REFERENCE TO RELATED APPLICATIONS

This application is a continuation of Patent Cooperation Treaty Application PCT/NL2014/050074, entitled “Groundbreaking Platform Technology for Specific Binding to Necrotic Cells”, to HQ MEDICAL (NETHERLANDS) B.V. filed on 6 Feb. 2014, which claims priority to Netherlands Patent Application Serial No. 2011274, filed 6 Aug. 2013, and to Patent Cooperation Treaty Application Nos. PCT/NL2013/050064 and PCT/NL2013/050067, both filed 6 Feb. 2013, and the specifications and claims thereof are incorporated herein by reference.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### COPYRIGHTED MATERIAL

Not Applicable.

### BACKGROUND OF THE INVENTION

1. Field of the Invention (Technical Field)

The invention relates to a system comprising a targeting molecule for binding necrotic cells; to the system for use as a medicament; to use of the system for diagnosis; to the system for use in treatment of diseases involving necrotic cell death, to a dosage comprising the system; to a method for determining localization of the composition within a sample using said system; and, to a method for treatment of cancers and/or diseases involving necrotic cell death using said system. In particular the invention relates to compositions targeting necrotic cells.

2. Description of Related Art

Prior art systems and methods involving targeting dead cells are not optimal and/or applicable:

a. Necrotic vs. Apoptotic Cell Death

Targeting of necrosis is specific and unique because necrosis only appears in pathological conditions of cell death due to an insufficient blood supply and thus a lack of oxygen, trauma or due to direct cytotoxic agents or other cancer treatments like radiotherapy or photo dynamic therapy. This is in contrast to apoptotic cell death, which occurs continuously during tissue turnover. Therefore apoptotic cell death, in contrast to necrotic cell death, is not useable for targeting diseases characterized by necrosis as found for instance in case of tumours (rapidly growing tumours spontaneously develop necrotic cores), trauma, infarcts, osteoarthiritis, diabetes, arteriosclerotic plaques, burns, certain bacterial infections, etc.

b. Antibodies Vs. Small Molecules

Our compounds are small molecules, cyanine based dyes, which penetrate deeply into necrotic tissue. Antibodies have been used for targeting necrosis (Tumour Necrosis Targeting) but have the disadvantage that they are too large to penetrate in the necrotic tissue.

c. Other Chemical Agents Vs. Cyanine Dyes

There are other chemical agents (for instance PDT-Agents) which are known for labelling necrotic cells (for instance Hypericine, Bacteriochlorophyll derivatives, Gadophrin-2A and bis-DTPA-NI). These agents have specific characteristics which make them less suitable for imaging and therapy of above mentioned diseases while they are (1.) difficult to produce chemically, they are (2.) photo sensitive (the compound will generate oxygen radicals and change their properties in case of exposure to light), (3.) the clearance from the body takes much longer (up to several days) and (4.) the compounds are relatively large which makes it more difficult to penetrate in necrotic tissue. Cyanine dyes are easy to produce chemically, are photo stable, are rapidly cleared from the body (3 to 24 hours) and deeply and easily penetrate in necrotic tissue.

Further details on background may be obtained from previously filed applications PCT/NL2013/050067 and PCT/NL2013/050064.

The following documents may be considered:

Madhuri Dasari et al., Organic Letters, 2010, 12 (15), 3300-3303, recite an imaging agent that detects necrotic tissue in vivo by binding extracellular DNA.

The present invention does not relate to DNA binding. DNA-binding molecuies are generally considered unsuitable for use in humans due the high chance that such molecules are mutagenic/carcinogenic.

Bryan A Smith et al., URL:http://etd.nd.edu/ETD-db/theses/available/etd-04132012-141111/unrestricted/SmithBryan042012D.pdf, recites in vivo imaging of cell death using fluorescent synthetic coordination complexes. In an aspect, the document recites a number apoptosis targeting molecules coupled to a cyanine dye.

The cyanine in these complexes is solely used as fluorescent reporter.

Epstein A L et al., Cancer Research, 1988, 48(20), 5842-5848 relates to a method for the detection of necrotic lesions in human cancers. Such relies on using antibodies as targeting moiety coupled to a dye as reporter.

The production, storage and use of antibodies is very difficult, expensive and high risk in terms of adverse effects.

Vera D et al., Nuclear Medicine and Biology, 2005, 32, 687-693, relates to a fluorescent probe for in vivo measurement of receptor biochemistry.

Uzgiris E E et al., Technology in Cancer Research and Treatment, 2006, 5 (4), 301-309, relates to a multimodal contrast agent for preoperative MR imaging and intraoperative tumor margin delineation.

Lia T et al, Bioorganic and Medicinal Chemistry Letters, 2010, 20, 7124-7126, relates to a low molecular weight near-IR probe for prostate cancer.

All of Vera D et al., Uzgiris E E et al and Lia T et al recite the use of cyanines as a reporter molecule coupled to various targeting moieties.

US 2012/088262 A1 relates to cyanine compounds, conjugates and methods for their use. Specifically said document recites amine-reactive cyanines.

It is noted that all proteins comprise amines. Amine reactive molecules will bind to any protein without large differences in affinity. In biological samples, such as blood and tissue, there is a vast amount of extracellular protein present. Selectivity for e.g. dead cells in the sample cannot therefore be achieved. In vivo, strong covalent binding to proteins will result in only very slow clearance from the body, which increases the chance of adverse effects.

Fang F et al., Spectrochimica Acta. Part A: molecular and biomolecular spectroscopy, 2006, 64 (3), 698-702, relates to determination of nucleic acids with near IR cyanine dyes using light-scattering techniques. Affinity of cyanines for DNA and RNA is identified.

DNA binding agents have limited use in humans as previously identified.

Hong Zheng et al., Analytical Biochemistry, 2003, 318, 86-90, relates to a dye binding assay using a long-wave-absorbing cyanine probe and describes the interaction of dye SHMC with various proteins.

Only analytical applications are identified.

Volkova K D et al., Journal of Biochemical and Biophysical Chemistry, 2007, 70, 727-733 relates to fluorescent probes for amyloid structures and in particular to cyanines capable of binding aggregates of the amyloid-β peptide.

Binding results from an interaction between the dye and features of the amyloid-β aggregate, not with individual amyloid-β peptides. Amyloid-β aggregates are extracellular.

US 2005/014216 relates using rhodamines and phalloidin capable of binding intracellular proteins as probes for hepatotoxicity.

Haibiao Gong et al., PLoS One, 2012, 7(3), e34003, XP055093071, relates to relates to near IR fluorescence imaging of mammalian cells and xenograft tumors with SNAP-Tag.

This document describes only applications of click chemistry (in this case via SNAP-Tag)

It is an object of the present invention to overcome one or more disadvantages of the compositions of the prior art and to provide alternatives to current compositions for diagnosis and treatment of cancers and other diseases involving necrotic cell death, without jeopardizing functionality and advantages.

### BRIEF SUMMARY OF THE INVENTION

The present invention relates to a system comprising a targeting molecule for binding necrotic cells, and one or more of an agent, a vehicle and an in the body non-reactive molecule according to claim 1.

The present system comprises at least two entities, the entities being joined, such as by a chemical bond, each entity serving a distinct function within the system. It is noted more than one agent may be present, as well as more than one vehicle, and more than one non-reactive molecule. As such, e.g., a combination therapy is feasible with the present system.

The present targeting molecule is very selective and very specific in binding to necrotic cells. As a result very low concentrations c.q. amounts of the present system may be used to provide advantageous effects thereof. It is noted that apoptosis specific probes, in contrast to necrosis specific probes will also target healthy tissue as apoptosis is involved in normal tissue turnover.

It has been found in earlier research by the present inventors that the targeting molecule specifically binds non-covalently to intracellular proteins such as actin, which are only available for the targeting molecule when the membrane integrity of a cell is lost, that is in case of a necrotic cell. For optimal targeting, the necrotic cells, are preferably in an early stage of necrosis, such as cells that have been dead for less than a few days, preferably less than half a day, such as a few hours, such as 2 hours, such as just dead cells. Characteristics of targeting molecules of the present invention, responsible for their effective targeting and/or safety, are that they are cell membrane impermeant, i.e. they cannot (significantly) cross the cell membrane of healthy cells; they are non-activated; they are capable of non-covalently binding to intracellular proteins (their target molecules); and, they are not capable/do not significantly bind to DNA (or RNA)

In case the system comprises an agent it may relate to an imaging compound, such as for use in an imaging technique, a therapeutic compound, in order to provide a therapeutic effect to necrotic cells or a part of a body (comprising necrotic cells) in need for such a therapeutic, such as a cancer, and a release factor, for releasing a further compound acting, e.g., as a stimulus.

In case the system comprises a vehicle the vehicle functions as a transport medium for transporting an entity to necrotic cells, or as above a part of a body. As such a minute amount of entity can be target and transported to a desired location in a body. The vehicle may transport the present agent, it may transport a relative to the agent, different entity, and combinations thereof.

In case the system comprises an in the body non-reactive molecule it is intended to have this molecule interact with a counterpart. Interaction may (further) relate to one or more of activation by an activator, support by a catalyst, generation of a medicament upon interaction, and release of a medicament upon interaction. It is noted that from a functional point of view molecule and counterpart may be interchanged.

The present system relates to non-toxic small molecules, having an ability to bind to necrotic cells and tissue. These molecules do not interact with DNA, i.e. these are not mutagenic. Typically the present system has a wide-spread biodistribution, cross the blood-brain barrier, do not bind to cell-surface proteins and are sufficiently stable.

In an example called ChemoTrace®, crucial time is saved during cancer treatment. ChemoTrace® instantly provides relevant data on the efficacy of the administered chemotherapeutic drug. An advantage of the present system is that it informs within 24-36 hours after a start of chemotherapy whether or not the therapy is effective. The use of ChemoTrace® will prevent patients from enduring heavy treatments without clinical benefits, which is regarded a major improvement in cancer therapy. The use of ChemoTrace® will result in cost savings.

It is noted that unfortunately a (positive) response rate to chemotherapy is limited to 20-35%. Further, the number of treatment cycles is typically limited to a maximum of four due to limitations of the human body. In other words it is crucial to identify a suitable therapy right from the start, before starting a cycle. By identifying at an early stage of treatment if a treatment is effective the treatment per se can be carried out if considered effective and can be skipped if considered not effective. In the case of a not effective treatment a second treatment can be started likewise. Such can be repeated a further number of times. Once a treatment is considered effective a cycle of chemotherapy can be initiated. It is noted that the identification can be repeated for every and any chemotherapy.

ChemoTrace® prevents over-treatment, saving on medicines used to prevent side-effects of cytotoxic drugs.

With the present system there is no need to culture harvested tumour cell, e.g., in order to perform tests, which can take a relatively long period of time e.g. a week. The necrotic cells are marked in their natural environment, which e.g. reduces a risk of human errors.

ChemoTrace® can be used to measure cytotoxic drug efficiency in vivo for all solid tumours which makes it an ideal system for a clinic. An advantage thereof is that in vivo relates to actual characteristics whereas in vitro may introduce deviations from the actual characteristics.

ChemoTrace® provides real-time data in the above respect, without a need to perform a puncture to harvest tumour cells. In this respect it is noted that by puncturing only a sample of a tumour is obtained. The obtained sample is not necessarily a true representation of the tumour. Further it is noted that a tumour is morphologically heterogeneous. Even further, the tumour may develop in morphological characteristics over time. The consequence hereof is that a punctured sample at the most gives an indication of characteristics of the tumour. The present invention does give the characteristics in real-time and with a high degree of certainty.

ChemoTrace® provides solid conclusions on the above points, such as through direct measurements, whereas prior art techniques only provide an indication of characteristics.

In an aspect, the present invention relates to a device which measures the effect on tumour cells as well on healthy tissue caused by radiotherapy, called Actinotrace®.

In an aspect, the present invention relates to a device which is a total body scan for detecting dead cells in the human body for prevention purposes, called NC-Scan®.

In an aspect, the present invention relates to delivery platform for treatment of cancer by targeting of necrotic cells in a tumour, called Ilumicore®.

In an aspect, the present invention relates to a platform to deliver active and regenerative compounds to a necrotic area caused by an ischemic event like a myocardial infarct or stroke, liver, kidney or other organ ischema, trauma, arthrosis or burn wounds, called Regenext®.

In an aspect, the invention relates to a non-nuclear alternative for a mammography scanner, called Mammolight®.

In an aspect, the present invention relates to a device for targeted delivery of (chemo) therapeutics in solid tumours, called Chemoclick®.

In an aspect, the invention relates to a targeted thermal therapy (Hyperthermic therapy), called Hyperthermys®.

Present diseases relate, amongst others to cancer, infarcted tissue of, e.g., heart and brain, osteoarthritis, neurological diseases such as Alzheimer's disease.

The present invention also relates to use of the composition as a medicament; for use of the system in a diagnostic method, use of the system for treatment of diseases involving necrotic cell death, to use of the composition for detecting and/or targeting necrotic cells in vivo and/or in vitro; to a dosage comprising said system; to a method for determining localization of the composition within a sample using said composition; and, to a method for treatment of cancers and/or diseases involving necrotic cell death using said composition.

Targeting molecules of the present invention have been found to bind to dead (necrotic) cells very selectively.

As mentioned earlier it has been found by the present inventors that necrotic cells and/or necrosis in general are attractive targets. Such relates to the observation that regions of necrotic cells are typically present in cancers (tumours), e.g., due to an insufficient blood supply and thus lack of oxygen, and in (Or result from) diseases involving necrotic cell death. it is noted that regions of necrotic cells are not typically found inside of healthy tissue. Examples of diseases involving necrotic cell death include infarcts or ischemic injury (i.e., Myocardial infarct, stroke, kidney, liver etc.), trauma (i.e., brain, muscle, bone) infections or inflammation (i.e., septic shock, rheumatoid arthritis, osteonecrosis), degenerative diseases (i.e., Alzheimer, Parkinson dementia), plaques (arteriosclerotic, amyloid) burn wounds, irradiation induced necrosis and diabetes.

With regards to cancer, once a tumour has been identified, further necrotic cell death can be induced intentionally (for instance by local irradiation, photo dynamic or local thermal therapy and/or focused ultrasound) in part of the tumour to provide a larger target for the composition. Furthermore, wherein the present composition is used for therapeutic purposes, the number of necrotic cells will increase as the therapy progresses thus resulting in dose amplification as a function of time.

A useful discussion on the classification of cell death can be found in Kroemer et al., Cell Death and Differentiation, 2005, 12, 1463. In the present application, necrotic cells are taken as cells whose plasma membrane has lost integrity. A person of skill in the art is able to determine whether a plasma membrane is intact i.e., integral, such as through using fluorescent dyes, such as using commercial amine reactive dyes. The selectivity of the composition of the present invention for dead cells, i.e., cells whose plasma membrane has lost integrity, can be demonstrated in in vitro tests as will be shown in the examples below. It is important to note that amine reactive dyes which bind to dead cells are useful only in vitro. In vivo, they would react with amines, e.g., of proteins or other compounds in blood, and would not be able to serve to target necrotic cells.

The term selectively indicates that the targeting compound has a higher affinity for necrotic cells than for healthy cells (thus the targeting molecules may target necrotic cells) Such can be determined in an in vitro assay as per the examples herewith, or, e.g., by flow cytometry; in both methods, co-staining may be used, e.g., using commercial live-dead cell staining kits. In simple terms, selective binding in the present invention indicates that for a given population of cells comprising necrosis and healthy cells the number of targeting molecules bound to necrotic cells is at least one order of magnitude higher than the number of targeting molecules bound to healthy cells, typically a few orders of magnitude, and preferably 6 or more orders of magnitude, such as 9 orders.

Non-activated cyanines are cyanines that are non-reactive towards, e.g., amines and thiols. Non-activated cyanines cannot significantly (reaction is thermodynamically unfavourable) bind to dead cells (functional groups of molecules thereof) through covalently attaching to amines, thiols or other reactive functional groups present on molecules found inside of cells. That is to say that selective binding to necrotic cells in the context of the present invention is not through covalent bonding, but rather through non-covalent binding via the cyanine core structure and not through the side chains to which the active groups are attached. The term activated cyanine is known to a person of skill in the art and includes, e.g., carboxylic acids activated as esters, N-hydroxysuccinimide esters, maleimides, acyl chlorides, SDS esters, etc. Non-activated cyanines include, e.g., cyanines comprising carboxylic acid functions, i.e., the carboxylic acid is not activated.

It is noted that activated. cyanines may be used in the formation of the composition, e.g., for the purpose of attaching an imaging and/or therapeutic compound to the cyanine; in the composition however, the cyanine is non-activated and will still bind to necrotic cells through the cyanine core structure.

Advantages of the present description are detailed throughout the description.

### DETAILED DESCRIPTION OF THE INVENTION

It is noted that examples given, as well as embodiments are not considered to be limiting. The scope of the invention is defined by the claims.

In a first aspect the present invention relates to a system comprising a targeting molecule for binding necrotic cells, the targeting molecule being selected from a cyanine or any other fluorescent dye, including Rhodamines, that specifically non-covalently binds to intracellular proteins. Proteins for binding may relate to fibrous proteins, such as 40 kDa proteins, 100 kDa proteins, such as tubulin, such as α-tubulin, β-tubulin, γ-tubulin, δ-tubulin and ε-tubulin, actin, such as G-actin, and F-actin, fibrous structural proteins, such as keratin, such as neutral, basic or acidic keratin, such as keratin 1-keratin 20, metalloenzymes, such as enolase, and lyase, CDC37, preferably tubulin, most preferably actin, isomers thereof, complexes thereof, and decay products thereof. These proteins are only available for the targeting molecule when the membrane integrity is lost, i.e., in case of a necrotic cell. Selectivity for binding necrotic cells can be determined, e.g., using the dry-ice dead-cell assay described below, in combination with fluorescent microscopy. Preferably there is least a 2-times higher affinity for necrotic cells compared to live cells, preferably at least 10-times, most preferably at least 1000-times, such as at least 1000000-times. Similarly, the affinity of the targeting molecule for one or more of the above proteins compared to an affinity for DNA is at least 2-times higher, preferably at least 10-times higher, most preferably at least 1000-times higher, such as at least 1000000-times higher, such as the targeting molecule does not and/or cannot bind to DNA, i.e., the targeting molecule does not significantly bind to DNA or RNA.

In an example, the targeting molecule is a non-reactive cyanine dye, according to FIG. 1 I, II and III, wherein n is an integer, such as nε[2,10], preferably nε[4,8], the chain L has up to n−1 double bonds, preferably n/2 double bonds, wherein sub-families II and III may comprise respectively one and two aromatic ring systems (A,B) signified by the curved line(s) C, wherein A,B are preferably selected each individually from benzene and naphthalene, wherein further groups R₅, R₆, R₇, and R₈, may be present, R₅, R₆, R₇, and R₈, are preferably selected each individually from H, and alkyl, such as methyl, ethyl, and propyl, preferably methyl, wherein the aromatic ring systems may comprise further functional groups R₁, R₂, and/or substituents, R₁, R₂, are preferably selected each individually from H, sulphonate, and sulphonamide, wherein the chain of alternating single and double bonds L may be interrupted by one or more partly and fully saturated ring structures, such as cyclopentene and cylcohexene, and combinations thereof, such as one or more cyclohexene rings, wherein the saturated ring structure may further comprise functional groups R₉, being selected from H, AA and BB, wherein R₁₀ is selected from, H, SO₃H, Cl, —N—C═O—(CH2)_{q}—Y₃ (q=1-6), —(CH2)ᵣ—Y₄ (r=1-6), Y₃ and Y₄ are each independently one of H, COOH, SO₃H, and CN, wherein the nitrogen atoms (N) may comprise further functional N-side groups R₃, R₄, wherein R₃, R₄ are preferably selected each individually from —(CH₂)ₘY, wherein Y is selected each individually from a carboxylic acid having 1-4 carbon atoms, a sulphonate group, CN, C≡C, and C═C, and salts thereof, wherein said N-side groups comprise m carbon atoms, such as mε[1,10], preferably mε[2,8], more preferably mε[3,7], most preferably m=4, 5, and 6, even more preferably at least one of m=4, 5, and 6, preferably one m=6, and the other m preferably is 4, 5 or 6, wherein said N-side groups comprise one or more functional groups on an end opposing the N, such as a carboxylic acid having 1-4 carbon atoms, an sulphonic group, and salts thereof, such as sodium and potassium salts, most preferably the functional group on the end comprises one or more double C—C bonds, preferably a carboxylate thereof, and/or wherein the targeting molecule is neutral or negatively charged.

Upon testing especially the above cyanines have been found to be very suitable, e.g., in terms of selectivity. Advantageously, cyanines of the invention having a negative charge have been found to bind preferentially to intracellular proteins in the presence of other cell components such as, e.g., DNA and RNA. That is to say, cyanines of the invention having a negative charge show in general no significant binding to DNA or RNA.

The targeting molecule is preferably selected from: HQ4, HQ5, HQ6, HQ7, ICG, CW 800, ZW800, L4, L7, L11, CY3, CY3b, CY3.5, CY5, CY5.5, CY7, Dy 676, Dy 681, Dy 731, Dy 751 and Dy 776; and, is most preferably selected from HQ4, HQ5, CW800 and ZW800. These compounds are presented in FIG. 2. Some of these compounds have for instance very good fluorescent properties. Such is rather unexpected, as a compound per se might have good fluorescent properties, but such may change upon binding to a further molecule, such as the present protein.

In an example, the agent is (one or more of the following (a), (b) and (c)): (a) an imaging compound selected from a group consisting a luminescent compound, a radioactive tracer, an MRI contrast or Spectroscopic agent, a RFA (radiofrequency ablation) agent, a PET agent, SPECT agent, ultrasound microbubble or contrast agent, opto-acoustic nanoparticles or contrast agent, CT contrast agent, a Raman agent or combinations thereof. In other words a suitable imaging compound may be used with the present system; surprisingly a wide range of such imaging compounds may be attached to the present system. As such a wide range of imaging techniques have become available. Such imaging techniques have been found to be very sensitive, i.e., small amounts of necrotic cells may be detected, very accurate, i.e., very small locations comprising necrotic cells may be identified, very reliable, and very confident, e.g., in terms of results. The agent may also be a therapeutic compound. As above the therapeutic compound can be brought to necrotic cells, using a small amount thereof, and yet providing a relative high amount thereof at an intended location, i.e., necrotic cells. The therapeutic compound (or one or more thereof) may be selected from a group consisting of chemo therapeutic agents, photo dynamic compounds, hyperthermic compounds (including photo thermal agents), immune modulating agents, proteins and peptides, nucleic acids (RNA- or DNA-based), medicaments (like antibodies), and/or combinations thereof. As such many therapies can be carried out, as well as combinations thereof. The agent may also be one or more release factors for stimulating tissue regeneration, such as growth factors and stem cells. As such repair and/or regeneration of tissues and cells are possible.

In an example, the vehicle is selected from a group consisting: a liposome, a dendrimer, a biodegradable particle, a micelle, a nanoparticle, an acoustic dissociable particle, a pH dissociable particle, a light dissociable particle, a heat dissociable particle, a chelating moiety and combinations thereof. Depending on the present system, e.g., in terms of further components being present, a suitable vehicle may be selected. Likewise a combination of vehicles may be selected. For instance a chelating moiety may be used for transporting In, F, Ga, Gd and Tc.

Vehicles may be considered substances that serve as mechanisms to improve the delivery and the effectiveness of drugs. Vehicles may be used for controlled-release in order to decrease drug metabolism, in order to prolong in vivo drug actions, and in order to reduce drug toxicity. Present vehicles are also used to increase effectiveness of drug delivery to a target site, e.g., of pharmacological action.

In an example, the in the body non-reactive molecule, which may be referred to as click A, is being capable of interacting with a counterpart, which may be referred to as click B. The non-reactive molecule is preferably one or more of a first Click-Chemistry component, a second Click-Chemistry component (complementary to the first Click-Chemistry component), and a component of a self-labelling protein tag such as of Snap-Tag. Preferably the Click-Chemistry components ‘click’ without requiring a catalyst such as copper (I)

In a second aspect the present invention relates to the system of the invention for use as a medicament. As mentioned above the present system may comprise a therapeutic compound. The system may therefor act as a medicament. It is preferred to use the present medicament for treating diseases, illnesses, etc. as identified throughout the application.

In a third aspect the present invention relates to use of the system of the invention in a diagnostic method, such as MRI, SPECT, RFA (Radiofrequency ablation), PET, CT, Raman spectroscopy, Ultrasound, Optical and Opto-acoustic. As the present system has superior characteristics for imaging, diagnosis by one or more of the aforementioned methods is preferred.

In a fourth aspect the present invention relates to the system of the invention for use in treatment of diseases involving necrotic cell death, including infarcts or ischemic injury, such as Myocardial infarct, stroke, and kidney, trauma, such as in a brain, a muscle, a bone, infections or inflammation, such as septic shock, rheumatoid arthritis, and osteonecrosis, degenerative diseases, such as Alzheimer, Parkinson dementia, plaques, such as arteriosclerotic, and amyloid, and Diabetes. Further examples of diseases for which the system may be used for treatment are included throughout the description.

In a fifth aspect the present invention relates to a dosage for detection and/or treatment of cancers and/or diseases involving necrotic cell death comprising an effective amount of the system of the invention.

In an example the dosage comprises an amount of 0.1-1000 nMole system/kg body weight, preferably 0.5-500 nMole system/kg body weight, more preferably 1-250 nMole system/kg body weight, even more preferably 2-100 nMole system/kg body weight, such as 5-50 nMole system/kg body weight; such may relate to a dosage of e.g. 0.01-1 mgram. The dosage preferably is provided in a physiological solution of 1-50 ml. Preferable a kit comprising some (1-50) dosages is provided.

In a sixth aspect the present invention relates to a method for determining localization within a sample, said sample comprising a population of cells comprising necrotic cells, comprising:

(a) providing a system according to the present invention;

(b) performing one or more measurements on the sample with at least a first suitable imaging technique providing a value or values; and

(c) analysing said value or values of the measurement to determine localization, optionally by comparison with a set of reference values. At least some of the above mentioned techniques are very suitable in this respect. The method may be carried out in vivo and in vitro.

In a seventh aspect the present invention relates to a method for treatment of cancers and/or diseases involving necrosis comprising:

(a) administering the composition of the present invention wherein the composition comprises one or more therapeutic compound (s);

(b) determining localization of the composition within the cancer and/or necrotic cell death; and

(c) activating release of at least the therapeutic compound(s) from the vehicle and/or activating the therapeutic compound(s). The treatment may be carried out in vivo.

The invention is further detailed by the Examples and accompanying figures, which are exemplary and explanatory of nature and are not limiting the scope of the invention. To the person skilled in the art it may be clear that many variants, being obvious or not, may be conceivable falling within the scope of protection, defined by the present claims.

### BRIEF SUMMARY OF FIGURES

FIGS. 1a-e show generic structures of three main sub-families of the present cyanine. Cyanine is a non-systematic name of a synthetic dye family belonging to polymethine group. Referring to the central carbon chain in FIG. 1; n is an integer, such as nε[2,10], preferably nε[4,8]. The chain L has up to n−1 double bonds, preferably n/2 double bonds. Sub-families II and III may comprise respectively one and two aromatic ring systems (A,B) signified by the curved line(s) C. A,B are preferably selected each individually from benzene and naphthalene. Groups R₅, R₆, R₇, and R₈, may be present. R₅, R₆, R₇, and R₈, are preferably selected each individually from H, and alkyl, such as methyl, ethyl, and propyl, preferably methyl. The aromatic ring systems may comprise further functional groups R₁, R₂, and/or substituents. R₁, R₂, are preferably selected each individually from H, sulphonate, and sulphonamide. The chain of alternating single and double bonds L may be interrupted by one or more partly and fully saturated ring structures, such as cyclopentene and cylcohexene, and combinations thereof, such as one or more cyclohexene rings. The saturated ring structure may further comprise functional groups R₉, R₂ being selected from H, AA and BB. R₁₀ is selected from, H, SO₃H, Cl, —N—C═O—(CH2)_{q}—Y₃ (q=1-6), —(CH2)ᵣ—Y₄ (r=1-6). Y₃ and Y₄ are each independently one of H, COOH, SO₃H, CN. The nitrogen atoms (N) may comprise further functional N-side groups R₃, R₄. R₃, R₄, are preferably selected each individually from —(CH₂)ₘY. Y is selected each individually from a carboxylic acid having 1-4 carbon atoms, a sulphonate group, CN, C≡C, and C═C, and salts thereof. N-side groups comprise m carbon atoms, such as mε[1,10], preferably mε[2,8], more preferably mε[3,7], most preferably m=4, 5, and 6, even more preferably at least one of m=4, 5, and 6, preferably one m=6, and the other m preferably is 4, 5 or 6. The N-side groups comprise one or more functional groups on an end opposing the N, such as a carboxylic acid having 1-4 carbon atoms, an sulphonic group, and salts thereof, such as sodium and potassium salts. Most preferably the functional group on the end comprises one or more double C—C bonds.

The term cyanine refers to any compound whose core-structure is that of sub-family I, II or III. The integer in names of cyanines such as Cy 3, Cy 5, Cy 7 etc. refers to the number of carbon atoms in the chain L. In an exemplary embodiment, the cyanine belongs to one of these families.

FIG. 2 shows a generic structure of a Rhodamine; R1 to R12 can be hydrogen or a functional group, examples of suitable functional groups include sulphonic acid groups, carboxylic acid groups, sulphonamides, alcohols, amines, esters, ethers, thiols, thio esters and combinations thereof. The term Rhodamine refers to any compound whose core-structure is that shown in FIG. 2.

FIGS. 3A-J give the structures of compounds referred to throughout the application. Where a counterion is shown, this may be for example H⁺, Na⁺ or K⁺.

FIGS. 4A-F and FIGS. 5A-E give preferred examples of the present targeting molecules.

FIGS. 6A-B, FIGS. 7A-D, FIG. 8, FIGS. 9A-C, FIG. 10 and FIG. 11 show results of experiments; these are detailed below.

FIG. 12 shows a schematic representation of NP-CW800.

FIG. 13 shows structure of DTPA-CO—NH-PEG-NH₂.

FIG. 14 shows structure of DTPA-CO—NH-PEG-HQ4.

FIGS. 15a-b show structures of two variants of Click A-HQ4-DTPA.

FIG. 16 shows structure of Click A-ZW800-DTPA.

FIGS. 17a-b show structures of two variants of Click A-ZW800-NOTA.

FIG. 18 shows structure of ClickA-ZW800 modified (at ammonium groups)-NOTA.

FIG. 19a shows structure of ZW800-linker-NOTA and Click A.

FIG. 19b shows structure of HQ4-linker-NOTA and Click A.

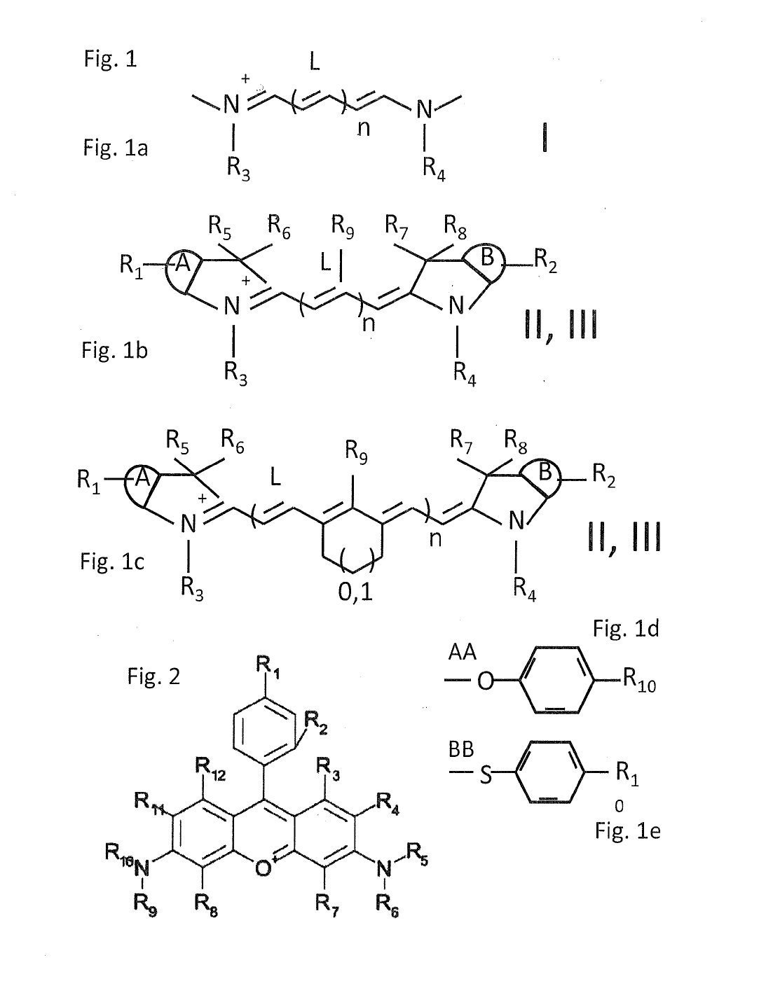

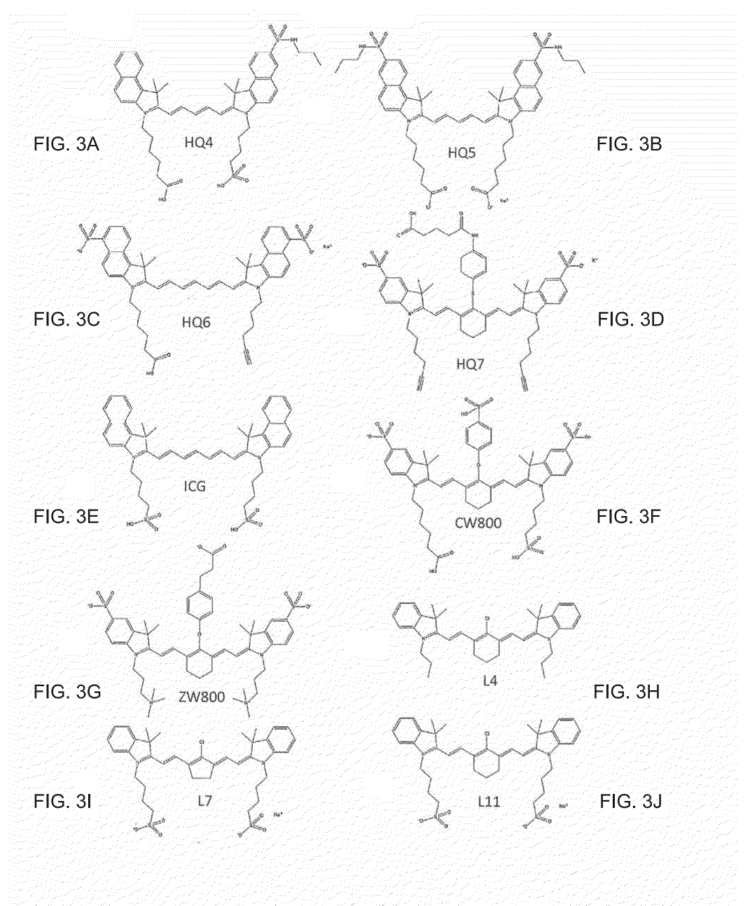

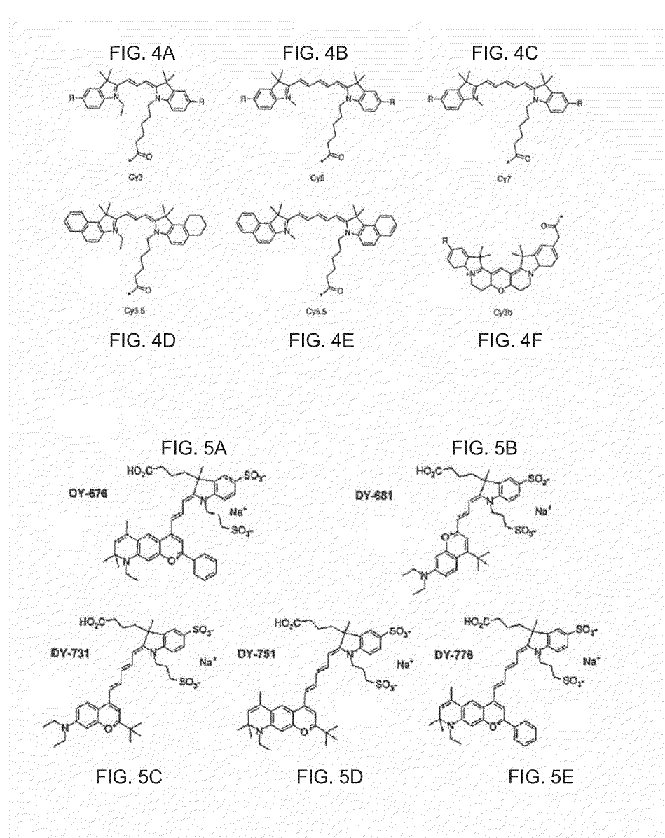

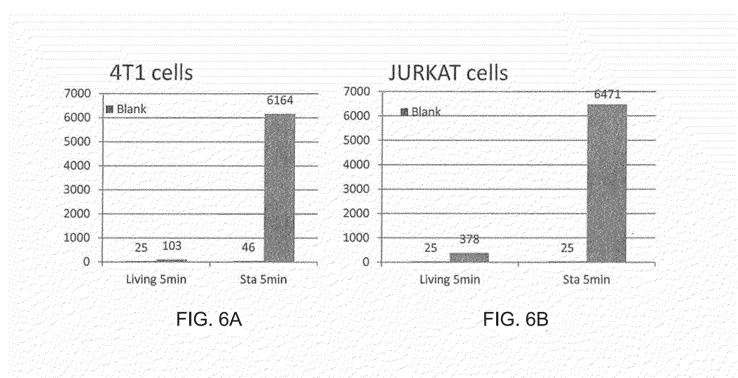

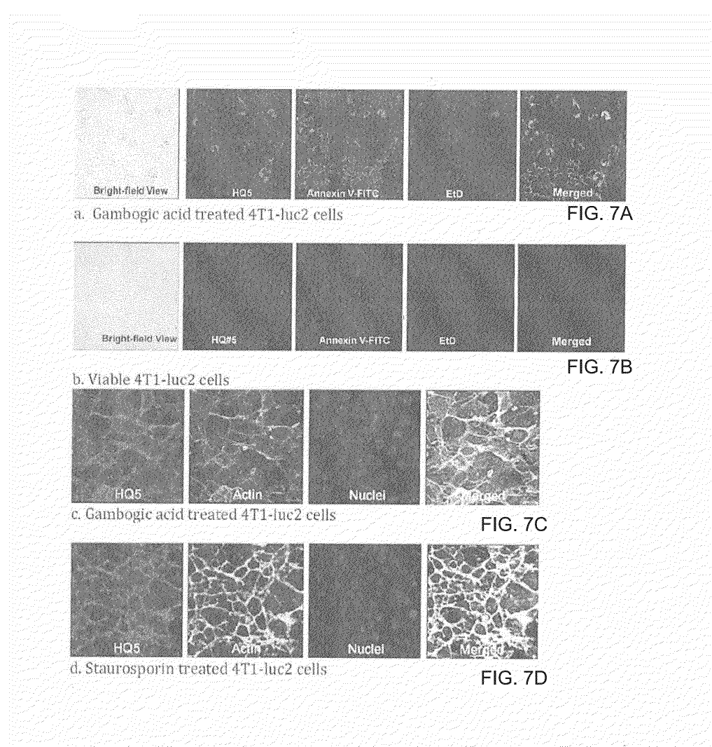

With reference to FIGS. 6-11:

Measurement Technology

In this investigation several existing and/or in-house developed techniques have been applied:

Fluorescence

[Wikipedia:] “Fluorescence is the emission of light by a substance that has absorbed light or other electromagnetic radiation of a certain wavelength. In most cases, the emitted light has a longer wavelength, and therefore lower energy, than the absorbed radiation.”

Measurements with fluorescence, so also in-vivo fluorescence, are hard to quantify. The measured intensity in a living creature or cell tissue is—besides the intrinsic brightness of the fluorescent compound itself and its concentration—also dependent of the physical properties of the chemical compound, i.e., the binding ratio with its surrounding proteins and the kind of interactions with them and the distribution of the substance through the tissue.

Finally, the sensitivity and specificity of the measurements are very strongly dependent of the experiment protocol.

Dry-Ice Dead Cell-Assay

Solid carbon dioxide in ethanol (−80° C.) is used to freeze the middle of a well of a 24 well-plate with a culture of living cells (and possibly other components). The procedure has been optimized by using a cooled block with pins that is held for a certain period of time (mostly approx. 15 sec.) to the bottom of the 24-well plate. The result is the freezing and consequent death of the cells in the middle surrounded by still living cells, enabling comparative studies between dead and living cells.

A medium containing compounds that can discriminate between living and dead cells is applied to the culture wells. After incubation the cell layer is washed and imaged.

Cryolesion Mouse Model

A small metal cylinder of 3 mm diameter is precooled in liquid nitrogen and applied to the parietal region of an anesthetized mouse's head for either 20 or 60 sec. After a certain period of time the dye is injected (in a certain concentration) in the tail vein. 3-24 hours after injection the mice can be imaged.

The selectivity of the composition of the present invention for necrotic cells, i.e., cells whose plasma membrane has lost integrity, is demonstrated in in vitro tests as will be shown in the examples below.

Matrigel

Matrigel is a gelatinous protein mixture secreted by specific mouse sarcoma cells. In this invention it is used as an endogenous matrix for dead cells that is implanted under the skin of a mouse. If these cells are also labelled it is confirmed that the dyes and constructs are distributed over the whole body, so also outside the bloodstream.

MSOT

Multispectral Optoacoustic Tomography is a technique for whole-body imaging of bio chemical markers in small animals. The absorption of light causes a small thermal expansion that genrates acoustic waves. These can be detected and converted in a three-dimensional image.

Proof of Concept

FIG. 6

Flow cytometry data using HQ5 on adherent (4T1) cells and non-adherent (JURKAT) cells treated or not treated with the cytotoxic agent Staurosporin. The probe only stains staurosporin treated cells, which have lost membrane integrity.

FIG. 7

Confocal microscopy of: a: gambogic acid (a necrosis inducing agent) treated cells and b: viable cells. C-D: Co-staining of a F-actin using phalloidin co-localized with HQ5 indicating actin binding of HQ5. (c: Gambogic acid treated cells and d: Staurosporin treated cells)

FIG. 8

To show the capacity of HQ5 to bind necrotic cells in vivo a local cryolesion of the brain was induced prior to HQ5 injection. In addition matrigel with or without necrotic cells was transplanted subcutaneously. HQ5 accumulates specifically in both the cryo-lesion and matrigel with necrotic cells, but not in the matrigel without necrotic cells.

FIG. 9

To show the capacity of HQ5 to bind necrotic cells in vivo in a more physiological model than cryolesions, Q 4-DTPA containing radioactive In-111 was injected in mice bearing tumours with: a necrotic core. A. Tumour specific HQ4 fluorescence was observed 24 hours after probe injection, lasting at least to 72 hours. B. A specific radioactive SPECT signal was detected 24 hours after probe injection, lasting at least to 72 hours. C. Ex vivo colocalization of both the fluorescent signal and radioactive signal with the necrotic core was observed.

FIG. 10

In order to investigate the possibility of using cyanine dyes to deliver PLGA nanoparticles to necrotic areas, PLGA nanoparticles coated with or without CW800 were injected into animals with a cryolesion of the brain. At low concentrations (diluted 2 times or more) CW800 coated PLGA nanoparticles accumulated selectively in the necrotic area whereas the non-coated control PLGA nanoparticles did not.

FIG. 11

Photodynamic therapy is known to cause necrosis. CW800 coated PLGA nanoparticles were injected 6 hours after photodynamic treatment of a 4T1 tumour on the left flank of the animal. A second tumour on the right flank remained untreated. The CW800 coated PLGA nanoparticles only accumulated at the site of the treated tumour.

### EXAMPLES

### Exemplary Systems of the Invention

### Example 1

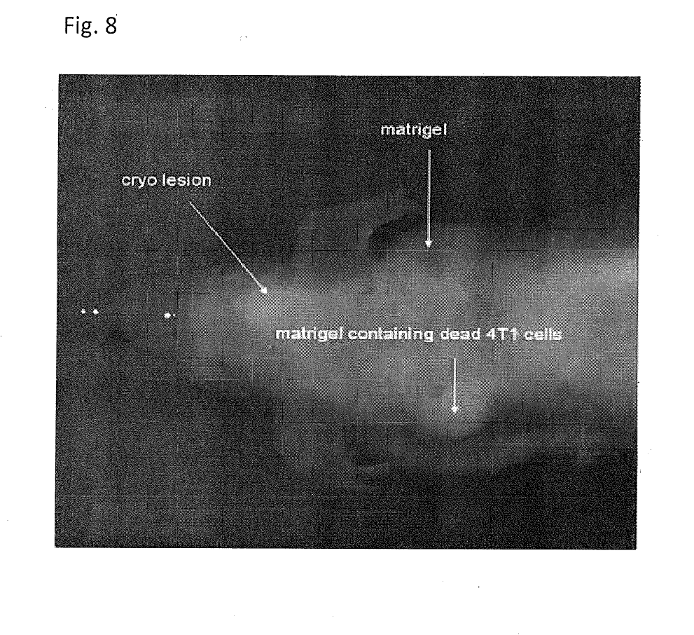

### With Reference to FIG. 12

PLGA NP Preparation.

PLGA NPs with entrapped near-infrared fluorescently labeled was prepared using an o/w emulsion and solvent evaporation-extraction method. In brief, 90 ing of PLGA in 3 mL of DCM containing the near-infrared (NIR) 700 nm (680R from LI-COR) (3 mg) was added drop-wise to 25 mL of aqueous 2% (w/v) PVA in distilled water and emulsified for 90 seconds using a sonicator (Branson, sonofier 250). A combination of lipids (DSPE-PEG(2000) amine (8 mg) and mPEG 2000 PE (8 mg)) were dissolved in DCM and added to the vial. The DCM was removed by a stream of nitrogen gas. Subsequently, the emulsion was rapidly added to the vial containing the lipids and the solution was homogenized during 30 seconds using a sonicator. Following overnight evaporation of the solvent at 4° C., the PLGA NPs were collected by ultracentrifugation at 6000 g for 30 min, washed three times with distilled water and lyophilized.

Conjugating CW800-NHS to the PLGA NPs.

CW800-NHS were conjugated to the DSPE-PEG (2000)-amine-containing PA NPs preparation. Subsequently, particles (50 mg) were dissolved in 0.5 mL of carbonated buffer (pH 8.5) and the activated CW800-NHS (1 mg) was added to the particles during 1 h at room temperature. Unbound CW800-NHS was removed by centrifugation (10000 g, during 10 min) and the PLGA NPs-CW800 was washed four times with PBS. The amount of CW800 on the particle surface was determined by Odyssey scanning (table 1).

Dynamic Light Scattering and Zeta-Potential.

Dynamic light scattering (DLS) measurements on all nanoparticles were performed on an ALV light-scattering instrument equipped with an ALV5000/60X0 Multiple Tau Correlator and an Oxxius SLIM-532 150 mW DPSS laser operated at a wavelength of 532 nm. A refractive index matching bath of filtered cis-decalin surrounded the cylindrical scattering cell, and the temperature was controlled at 21.5±0.3° C. using a Haake F3-K thermostat. For each sample the auto-correlation function, g₂(τ), was recorded ten times at a detection angle of 90°. For each measurement the diffusion coefficient, D, was determined using the 2^{nd} order cumulant and the correspond ng particle diameter was calculated assuming that the particles were spherical in shape (see table 1). Zeta potential of all nanoparticles was determined on a Malvern ZetaSizer 2000 (UK)

| | | | | | | Ligands (# |
|---|---|---|---|---|---|---|
| | Nanosphere | | | Zeta | Targeting moiety | ligands/NP) |
| NPs with and without | size ± | | Width | potential ± | CW800 ug/mg | (Experimental |
| targeting moiety | S.D. (nm) | PDI | (nm) | S.D. (mV) | PLGA) (w/w) | values) |
| PLGA-NP (NIR-680)-PEG- | 214.0 ± 12.3 | 0.204 | 75.6 | −25.4 ± 1.60 | — | — |
| Not | | | | | | |
| PLGA-NP (NIR-680)-PEG- | 216.2 ± 10.5 | 0.196 | 73.2 | −41.5 ± 3.25 | 0.25 ug | 866 |
| CW800 | | | | | | |

### Example 2

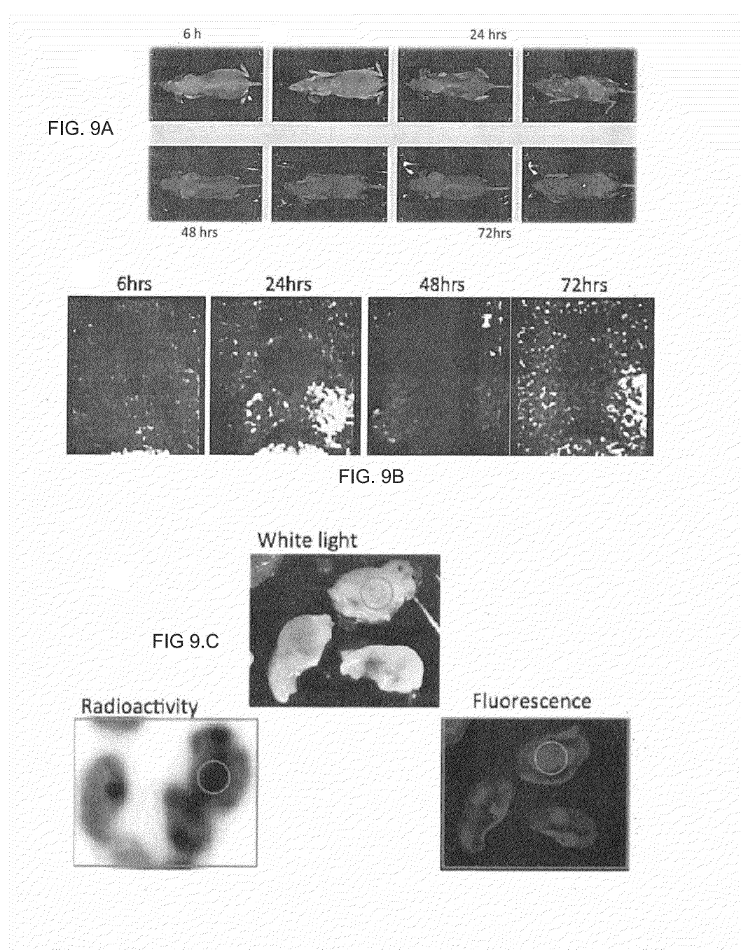

### With Reference to FIG. 13

Synthesis of DTPA-PEG-NH2.

DTPA containing PEG amine link (4,7,10-Trioxa-1,13-tridecanediamine, indicated as PEG-NH₂ in the formula (DTPA-PEG-NH2) was synthesised on Cl-TrtCl resin. Thus, Fmoc-PEG amine was incorporated on Cl-TrtCl resin (CTC resin) by reacting 3 eq. Fmoc-PEG amine in presence of 6 eq. DIEA in DCM during overnight at room temperature. Final loading was measured by Fmoc quantification: value obtained was around 0.8 mmol/g. Fmoc group removal was carried out with piperidine-DMF (1:5) (1×1 min, 2×10 min). Next, the DTPA-(tetra-tBu ester)-COOH (2 eq.) was coupled using DIPCDI (2 eq.) and HOBt (2 eq.) in DMF during overnight. After coupling overnight the ninhydrin test was negative. Later on, DTPA-(tetra-tBu ester)-CO—NH-PEG-CTC-resin cleavage and deprotection was performed in two steps. DTPA-(tetra-tBu ester)-CO—NH-PEG-CTC-resin was treated with 1% TFA in DCM for 10 times for 1 min each time. Excess DCM was removed using vacuum and side chain protecting groups were removed using 95% TFA, 2.5% TIS and 2.5% water. DTPA-CO—NH-PEG-NH2 was precipitated with cold MTBE after TFA removal under a N₂ stream. The DTPA-CO—NH-PEG-NH2 was dissolved in water and lyophilized to obtain the final product. The desired DTPA-CO—NH-PEG-NH2 was 85.0% in yield with a purity of 90.6% as analysed by HPLC (tR 2.28 min). HPLC-MS, m/z calc.: 523.25 for C₂₀H₃₇N₅O₁₁. Found: 524.5.28 [M+1]+ and MALDI-TOF Found 524.2 [M+1]+ 546.3 [M+Na].

### Example 3

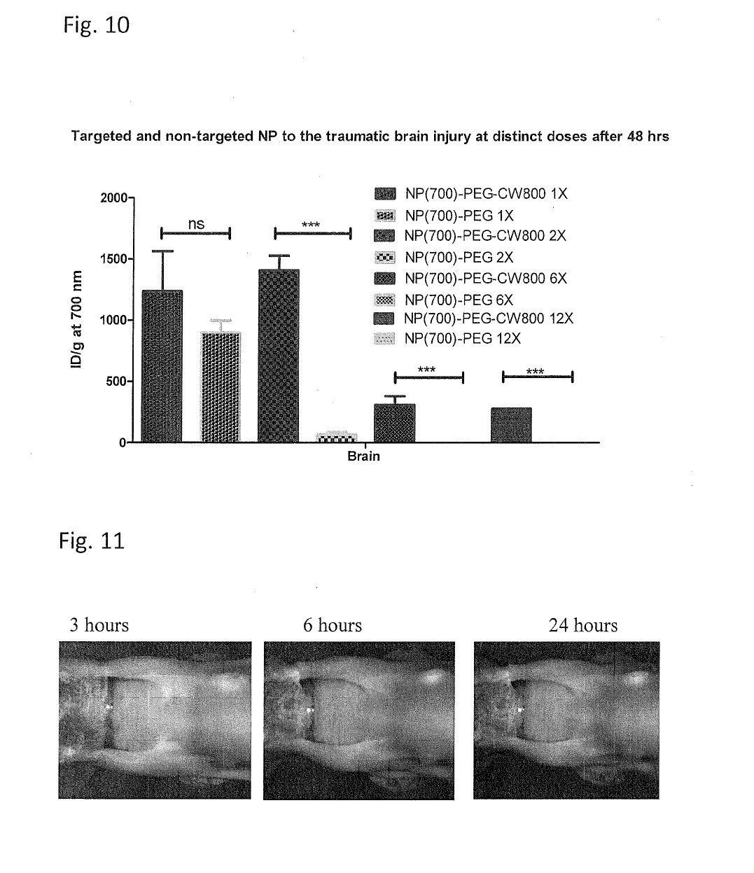

### With Reference to FIG. 14

Synthesis of DTPA-CO—NH-PEG-NH2-HQ4.

HQ4-NHS (8.3×10⁻⁴ mmol, 1 mg) dissolved in 50 μL of DMSO was added to DTPA-CO—NH-PEG-NH2 (2.8×10⁻³ mmol, 2 mg) dissolved in 200 μL of DMSO containing 5 μL DIEA and stirred overnight at room temperature. Later on, the complex DTPA-CO—NH-PEG-HQ4 was purified by RP-HPLC. The desired Dota-PG-HQ5 was 50.5% in yield with a purity of 98% as analysed by HPLC (tR 5.34). HPLC-MS, m/z calc.: 1331.59 for C₆₆H₉₀N₈O₁₇S₂. Found: 1332.0 [M+1]+ and MALDI-TOF Found 1331.9 [M+1]+ 1353.9 [M+Na].

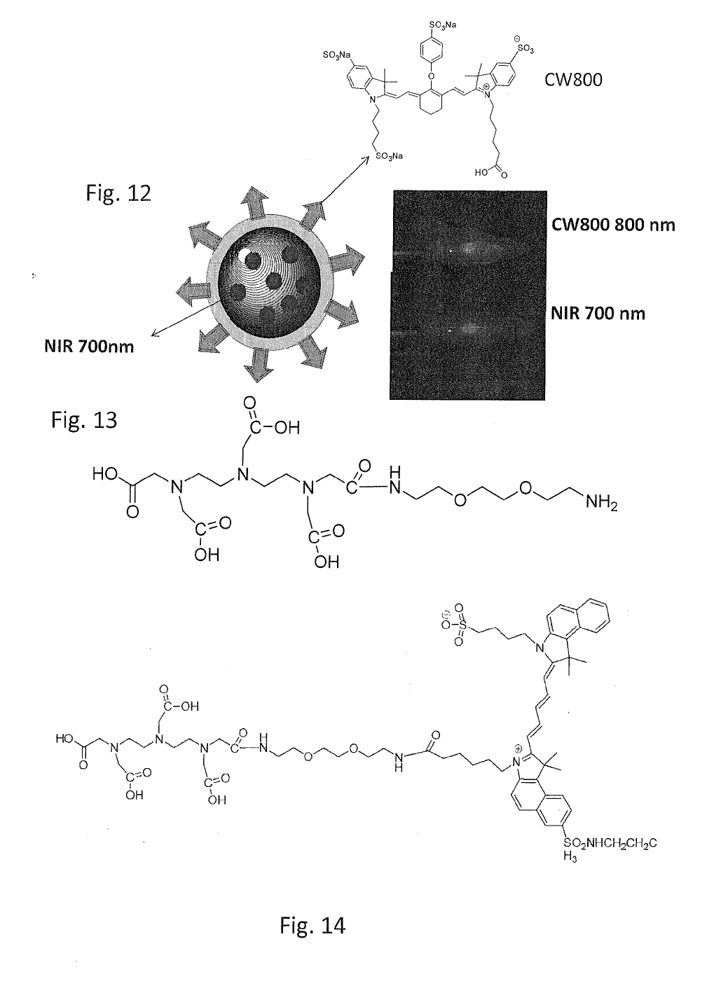

Further Examples of systems according to the invention are shown in FIGS. 15-19.

FIGS. 15a and 15b—variants of Click A-HQ4-DTPA

Click A=2-cyanobenzothiazole
Vehicle=Diethylenetriaminepentaacetic acid (DTPA)

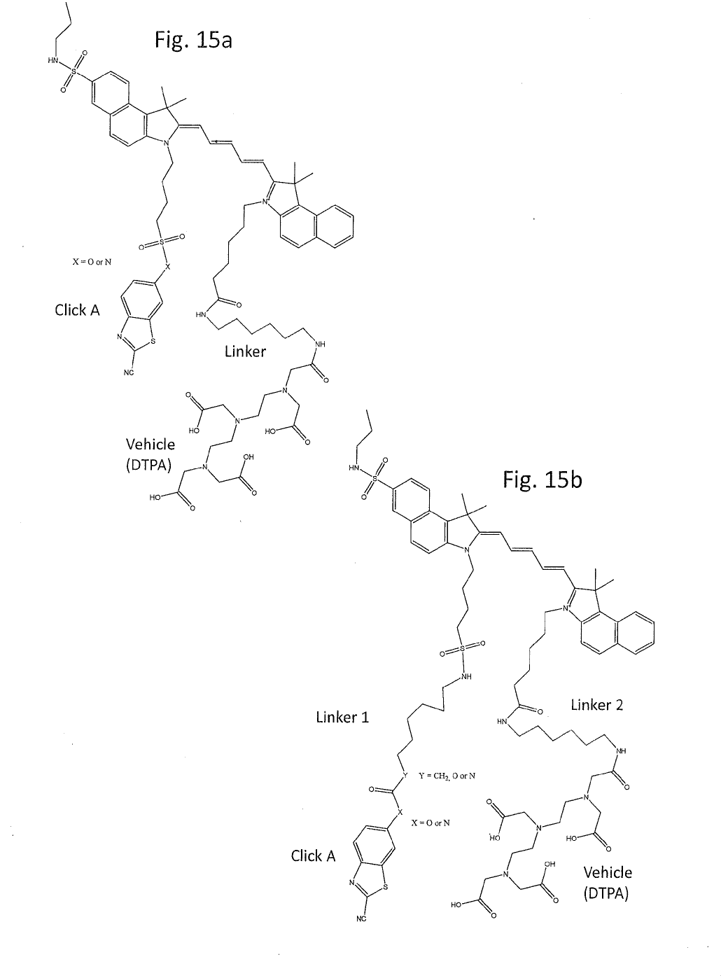

FIG. 16—ClickA-ZW800-DTPA

Click A=2-cyanobenzothiazole
Vehicle=Diethylenetriaminepentaacetic acid (DTPA)

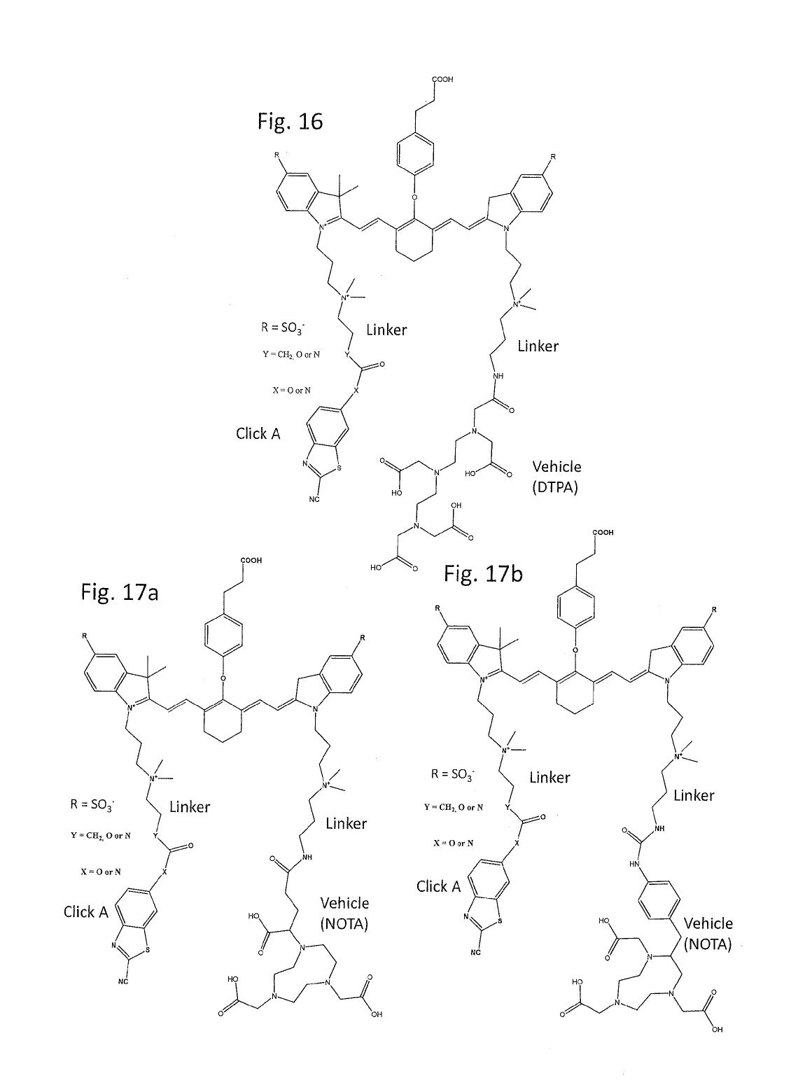

FIGS. 17a and 17b—variants of ClickA-ZW800-NOTA

Click A=2-cyanobenzothiazole
Vehicle=2,2,2-(1,4,7-triazanonane-1,4,7-triyl)-triacetate (NOTA)

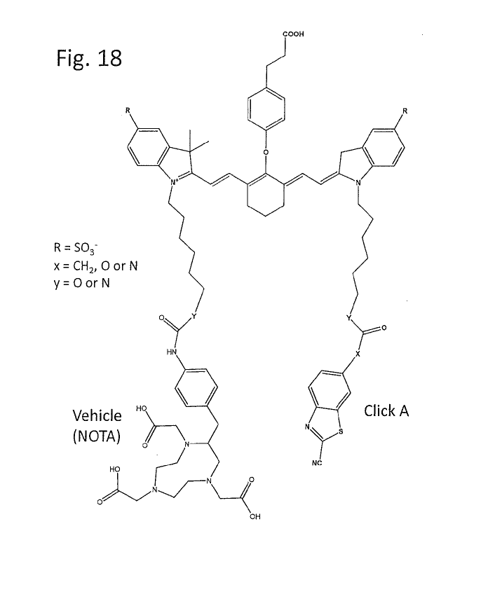

FIG. 18—ClickA-ZW800-modified (at ammonium groups)NOTA

Click A=2-cyanobenzothiazole
Vehicle=2,2,2-(1,4,7-triazanonane-1,4,7-triyl)triacetate (NOTA)

It is noted that whilst in examples 16-18, R═SO₃⁻ (i.e., the cyanine is compound ZW800), R may also be another moiety, such as, e.g., H, alkyl etc., provided the resulting cyanine falls within the scope of one or more of the claims.

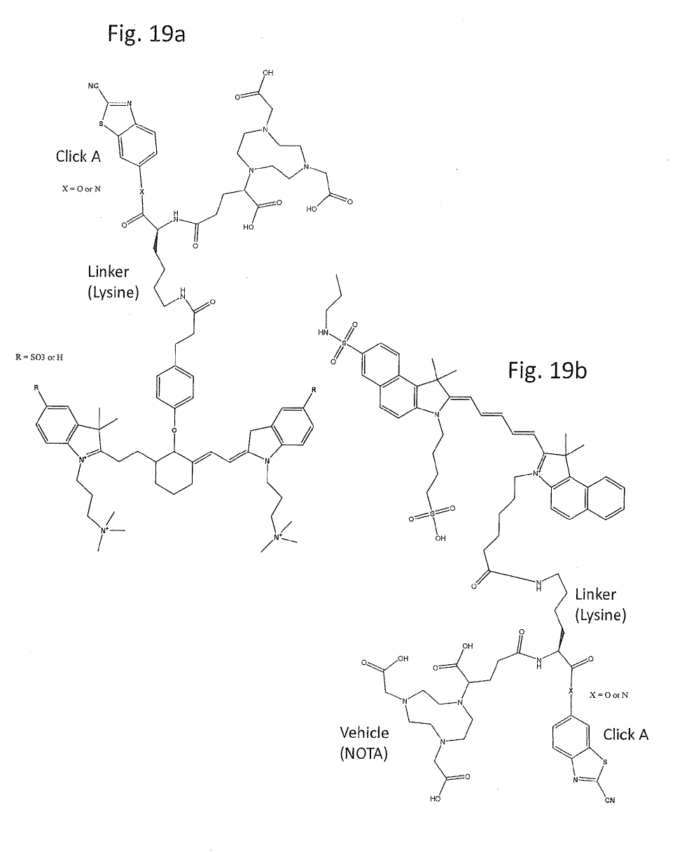

FIG. 19a—ZW800-linker-NOTA and Click A

Click A=2-cyanobenzothiazole
Vehicle=2,2,2-(1,4,7-triazanonane-1,4,7-triyl)triacetate (NOTA)

FIG. 19b—HQ4-linker-NOTA and Click A

Click A=2-cyanobenzothiazole
Vehicle=2,2,2-(1,4,7-triazanonane-1,4,7-triyl)triacetate (NOTA)

As an alternative to the systems of claims 15-18, wherein the vehicle and click A (in the body non-reactive molecule being capable of interacting with a counterpart) are provided on distinct first and second functional groups of the cyanine, both the vehicle and click A (in the body non-reactive molecule being capable of interacting with a counterpart) may be linked to a single functional group of the cyanine using a bifunctional linker, such as through lysine as shown in FIG. 19. In FIG. 19a, click A and NOTA are linked through the carboxylic acid group of ZW800. A similar strategy may be applied to link via a side-arm i.e. the nitrogen of an ammonium group.

A person skilled in the art will realise that other combinations of targeting molecule and: (a) agent, (b) vehicle and/or (c) in the body non-reactive molecule being capable of interacting with a counterpart, are possible e.g. Click A-HQ4-NOTA, Click A-HQ5-NOTA DTPA, Click A-CW800-NOTA/DTPA, Click A-ZW800-NOTA/DTPA etc. NOTA and DTPA are examples of chelating ligands suitable for transporting an agent, such as a metal ion.

Glossary

4T1-cells Breast cancer cells from murine origin.

4T1-luc2 Same cells, but genetically modified to have luciferase-2, an enzyme that can convert luciferine (luc) accompanied by the radiation of light. So, after administration of luc these cells can be detected.
BSA Bovine Serum Albumin, a water soluble protein from bovine serum used for all kinds of biological applications, among others to study the binding of a substance to proteins.

### CRO Contract Research Organization

FACS Fluorescence Activated Cell Sorting, a technique where the fluorescence of a flow of individual cells can be measured.
FCS Fetal Calf Serum, a serum from mentioned origin, with comparable application as BSA Lysate A fluid containing the contents of cells that are broken down and where the integrity and organization is all gone.
MALDI-TOF Matrix-Assisted Laser Desorption/Ionization—Time-Of-Flight is an chemical analytical technique to determine the molecular mass of a chemical entity. MALDI is a ‘soft’ ionization technique, while TOF is a detection technology particularly suitable for large (heavy) molecules.
MRI Magnetic Resonance Imaging, a medical imaging technique, making use of the principle of NMR.
MTS-assay a calorimetric assay that measures the viability of the cell. The higher the signal, the more viable the cells are.
MSOT Multispectral Optoacoustic Tomography, see paragraph 3.2.

### NIRF Near InfraRed Fluorescence

NMR Nuclear Magnetic Resonance, a physical phenomenon in which atoms with specific properties (i.e. being magnetic, having a spin) can absorb and re-emit radiation of an atom-specific wavelength.
PDT Photo dynamic therapy is a form of phototherapy using nontoxic light-sensitive com-pounds that are exposed selectively to light, whereupon they become toxic to targeted malignant and other diseased cells.

### PET Positron Emission Tomography

PKPD PharmacoKinetics and PharmacoDynamics studies how the substance behaves in a living body (bioavailability, distribution, metabolism) and how it interacts with the biological target (binding, residence time)
SDS-PAGE Sodium Dodecyl Sulphate PolyacrylAmide Gel Electrophoresis is a technique to separate mixtures of proteins into individual proteins (or mixtures with less components)
SPECT Single-Photon Emission Computed Tomography is an imaging technique using gamma rays. It is able to provide true 3D information. This information is typically presented as cross-sectional slices through the patient, but can be freely reformatted or manipulated as required. [Wikipedia]
TUNEL Terminal deoxynucleotidyl transferase dUTP nick end labelling is a method for detecting DNA fragmentation by labelling the terminal end of nucleic acids [Wikipedia]. It is used as a labelling tool of necrotic tissue.

What is claimed is:

1. A system comprising a targeting molecule for binding necrotic cells, the targeting molecule being a cyanine that is cell membrane impermeant, is non-activated, is capable of non-covalently binding to intracellular proteins wherein the targeting molecule is neutral or negatively charged, wherein the targeting molecule is selected from: HQ4, HQ5, HQ6, HQ7, ICG, ZW800, L7, L11, CY3, CY3b, CY3.5, CY5, Dy 676, Dy 681, Dy 731, Dy 751 and Dy 776; and does not significantly bind to DNA,
wherein the system further comprises one or more of:
(i) an agent selected from an imaging compound, a therapeutic compound, and a release factor;
(ii) a vehicle that is effective for transporting an/the agent; and
(iii) an in the body non-reactive molecule being capable of interacting with a counterpart.

2. A system according to claim 1, wherein the targeting molecule is selected from ZW80, HQ4, HQ5, HQ6, and HQ7.

3. A system according to claim 1, wherein the agent is:
(a) an imaging compound selected from a group consisting a luminescent compound, a radioactive tracer, an MRI contrast or Spectroscopic agent, a PET agent, a RFA (Radio Frequency Ablation) agent, a SPECT agent, an ultrasound microbubble or contrast agent, opto-acoustic nanoparticles or contrast agent, CT contrast agent, a Raman agent, and combinations thereof; and/or
(b) a therapeutic compound selected from a group consisting of chemo therapeutic agents, photo dynamic compounds, hyper-thermic compounds (including photo thermal agents), immune modulating agents, proteins and peptides, nucleic acids (RNA- or DNA-based), medicaments (like antibodies), and combinations thereof; and/or
(c) a release factor for stimulating tissue regeneration, such as growth factors and stem cells.

4. A system according to claim 1, wherein the vehicle is selected from a group consisting: a liposome, a dendrimer, a biodegradable particle, a micelle, a nanoparticle, an acoustic dissociable particle, a pH dissociable particle, a light dissociable particle, a heat dissociable particle, a chelating moiety, and combinations thereof.

5. A system according to claim 1, wherein the in the body non-reactive molecule being capable of interacting with a counterpart is selected from: a first Click-Chemistry component, a second Click-Chemistry component being complementary to the first Click-Chemistry component, and a component of a self-labelling protein tag such as of Snap-Tag.

6. A system comprising a targeting molecule for binding necrotic cells, the targeting molecule being a cyanine that is cell membrane impermeant, is non-activated, is capable of non-covalently binding to intracellular proteins wherein the targeting molecule is neutral or negatively charged, and does not significantly bind to DNA,
wherein the system further comprises one or more of:
(i) an agent selected from an imaging compound, a therapeutic compound, and a release factor;
(ii) a vehicle that is effective for transporting an/the agent; and
(iii) an in the body non-reactive molecule being capable of interacting with a counterpart for use as a medicament.

7. Use of a system comprising a targeting molecule for binding necrotic cells, the targeting molecule being a cyanine that is cell membrane impermeant, is non-activated, is capable of non-covalently binding to intracellular proteins wherein the targeting molecule is neutral or negatively charged, and does not significantly bind to DNA,
wherein the system further comprises one or more of:
(i) an agent selected from an imaging compound, a therapeutic compound, and a release factor;
(ii) a vehicle that is effective for transporting an/the agent; and
(iii) an in the body non-reactive molecule being capable of interacting with a counterpart, in a diagnostic method selected from MRI, SPECT, PET, RFA (Radio Frequency Ablation), CT, Raman spectroscopy, Ultrasound, Optical and Opto-acoustic.

8. A system comprising a targeting molecule for binding necrotic cells, the targeting molecule being a cyanine that is cell membrane impermeant, is non-activated, is capable of non-covalently binding to intracellular proteins wherein the targeting molecule is neutral or negatively charged, and does not significantly bind to DNA,
wherein the system further comprises one or more of:
(i) an agent selected from an imaging compound, a therapeutic compound, and a release factor;
(ii) a vehicle that is effective for transporting an/the agent; and
(iii) an in the body non-reactive molecule being capable of interacting with a counterpart for use in the treatment of diseases involving necrotic cell death, including infarcts or ischemic injury, such as Myocardial infarct, stroke, and kidney, trauma, such as in a brain, a muscle, a bone, infections or inflammation, such as septic shock, rheumatoid arthritis, and osteonecrosis, degenerative diseases, such as Alzheimer, Parkinson dementia, plaques, such as arteriosclerotic, and amyloid, and Diabetes.

9. Dosage for detection and/or treatment of cancers and/or diseases involving necrotic cell death comprising an effective amount of a system comprising a targeting molecule for binding necrotic cells, the targeting molecule being a cyanine that is cell membrane impermeant, is non-activated, is capable of non-covalently binding to intracellular proteins, wherein the targeting molecule is neutral or negatively charged, and does not significantly bind to DNA,
wherein the system further comprises one or more of:
(i) an agent selected from an imaging compound, a therapeutic compound, and a release factor;
(ii) a vehicle that is effective for transporting an/the agent; and
(iii) an in the body non-reactive molecule being capable of interacting with a counterpart.

10. Dosage according to claim 9, comprising an amount of 0.1-1000 nMole system/kg body weight and/or is provided in a physiological solution of 1-50 ml.

11. A method for determining localization within a sample, said sample comprising a population of cells comprising necrotic cells, comprising:
(a) providing a system comprising a targeting molecule for binding necrotic cells, the targeting molecule being a cya-nine that is cell membrane impermeant, is non-activated, is capable of non-covalently binding to intracellular proteins, wherein the targeting molecule is neutral or negatively charged, and does not significantly bind to DNA,
wherein the system further comprises one or more of:
(i) an agent selected from an imaging compound, a therapeutic compound, and a release factor;
(ii) a vehicle that is effective for transporting an/the agent; and
(iii) an in the body non-reactive molecule being capable of interacting with a counterpart;
(b) performing one or more measurements on the sample with at least a first suitable imaging technique providing a value or values, and;
(c) analysing said value or values of the measurement to determine localization, optionally by comparison with a set of reference values.

12. A method for treatment of cancers and/or dis-eases involving necrotic cell death comprising:
(a) administering a system comprising a targeting molecule for binding necrotic cells, the targeting molecule being a cyanine that is cell membrane impermeant, is non-activated, is capable of non-covalently binding to intracellular proteins, wherein the targeting molecule is neutral or negatively charged, and does not significantly bind to DNA,
wherein the system further comprises one or more of:
(i) an agent selected from an imaging compound, a therapeutic compound, and a release factor;
(ii) a vehicle that is effective for transporting an/the agent; and
(iii) an in the body non-reactive molecule being capable of interacting with a counterpart, wherein the composition com-prises one or more therapeutic compound(s)
(b) determining localization of the composition within the cancer and/or necrotic tissue
(c) activating release of at least the therapeutic com-pound(s) from the vehicle and/or activating the therapeutic compound(s).
